(19) United States
(12) Patent Application Publication
(10) Pub. No.: US 2012/0129822 A1
(43) Pub. Date: May 24, 2012

(54) COMPOSITION CONTAINING DIOSGENIN AND USE THEREOF TO IMPROVE AT LEAST ONE OF COGNITIVE DEFICITS ASSOCIATED WITH MENOPAUSAL SYNDROME

(75) Inventors: Ying-Jui HO, Taichung City (TW); Jian-Horng CHEN, Nantou County (TW)

(73) Assignees: Ying-Jui HO; TL & GM Chemical Company; Chung Shan Medical University

(21) Appl. No.: 13/111,844

(22) Filed: May 19, 2011

(30) Foreign Application Priority Data
Nov. 18, 2010 (TW) 099139715

(51) Int. Cl. A61K 31/58; A61P 25/28

(52) U.S. Cl. 514/173

(57) ABSTRACT

Disclosed herein is a composition for improving at least one of cognitive deficits associated with menopausal syndrome, which includes diosgenin. Also disclosed herein is a method for improving at least one of cognitive deficits associated with menopausal syndrome in a subject via administering the aforesaid composition.

### CROSS-REFERENCE TO RELATED APPLICATION

This application claims priority of Taiwanese Application No. 099139715, filed on Nov. 18, 2010.

### BACKGROUND OF THE INVENTION

1. Field of the Invention

This invention relates to a composition containing diosgenin and the use thereof to improve at least one of cognitive deficits associated with menopausal syndrome.

2. Description of the Related Art

Menopause is related to a rapid decline in circulating sex hormones of a female, and refers to the transition time from the beginning of the gradual failure in the ovarian function to the cessation of menstruation. Postmenopause refers to the time after the complete loss of ovarian function. Menopausal or postmenopausal females normally have menopausal syndrome. Generally speaking, symptoms of menopausal syndrome can be classified into three categories, i.e., physiological symptoms, psychological symptoms, and cognitive deficits. The physiological symptoms include hot flushes, vaginal dryness, fatigue, headaches, weight gain, skin aging, and osteoporosis. Examples of the psychological symptoms are anxiety and depression. The cognitive deficits include mental confusion, decline in memory, learning, and recognition abilities, and inability to cope with stress.

Menopausal syndrome is mostly treated with hormone replacement therapy (HRT) in which a pharmaceutical composition containing one or more sex hormones is administered to a patient. HRT is able to boost the level of sex hormone in menopausal or postmenopausal females. However, long-term administration or overdose of sex hormones may lead to breast cancer. In contrast with sex hormones, phytoestrogens (e.g., isoflavone), which have effects similar to those of human estrogens, are relatively safe. Consequently, phytoestrogens are more preferably used for HRT.

Since dioscorea contains plenty of phytosteroids, the same has been used as a Chinese medicine for improving gastrointestinal, sensory, memory, and sexual-related functions, and for ameliorating hot flushes and frequent urination in postmenopausal females. In addition, dioscorea has been proven to have anti-osteoporotic, anti-diabetic, and anti-hypercholesterolemic activities.

In Annals of General Psychiatry (2007), 6:21-28, Y. J. Ho et al. investigated the effects of dioscorea on emotional behavior and interleukin-2 (IL-2) levels in brains of ovariectomized (OVX) rats, and obtained the results revealing that the dioscorea treatment is capable of reversing OVX-induced anxiety and changes in the neuroimmunological function of the cortex.

Diosgenin is the major steroidal saponin in dioscorea, and is similar to sex hormones in terms of the chemical structure. Therefore, diosgenin has been widely used as a precursor of steroidal hormones such as progesterone, estrogen, testosterone, and cortisone. Steroidal hormones can be prepared by subjecting diosgenin to in vitro chemical modification.

In recent years, diosgenin has been developed to treat menopausal syndrome. For instance, in Biomed Sci Instrum. (2001), 37:281-286, K. Higdon et al. induced osteoporosis in OVX rats, and treated the OVX rats thus having clinical signs of osteoporosis with the sustained delivery of diosgenin (DG), dehydroepiandrosterone (DHEA) or estrogen (E). The results show that bone loss after ovariectomy was significantly reduced via supplementation with sustained levels of DG, DHEA, and E without inducing side effects on other body organs.

In Steroids (2009), 74:504-511, Y. Tada et al. studied the effect of diosgenin on skin aging during menopause and the safety regarding the use of diosgenin to treat skin aging during menopause. The results thus obtained indicate that: the administration of diosgenin is able to restore keratinocyte proliferation in aged skin, thereby being capable of improving epidermal thickness of OVX mice (i.e., an animal model of menopause) without altering the degree of fat accumulation. Furthermore, diosgenin does not accelerate the tumor growth in breast cancer-burdened mice.

Through research, the applicants have found that diosgenin is effective in improving at least one of cognitive deficits associated with menopausal syndrome (e.g. decrease in memory, learning, and recognition abilities).

### SUMMARY OF THE INVENTION

Therefore, according to a first aspect, this invention provides a composition for improving at least one of cognitive deficits associated with menopausal syndrome, which comprises diosgenin.

According to a second aspect, this invention provides a method for improving at least one of cognitive deficits associated with menopausal syndrome in a subject, which comprises administering to the subject the aforesaid composition.

### BRIEF DESCRIPTION OF THE DRAWINGS

The above and other objects, features and advantages of this invention will become apparent with reference to the following detailed description and the preferred embodiments taken in conjunction with the accompanying drawings, in which:

FIG. 1 is a schematic perspective view of an elevated plus-maze;

FIG. 2(A) is a schematic top view to illustrate an open box and three objects A1, A2, A3 disposed therein;

FIG. 2(B) is a schematic top view illustrating that the object A3 in the open box as shown in FIG. 2(A) is replaced with an object B; and

FIG. 3 shows exploration time spent on object A3 in a second trial of an object recognition test and exploration time spent on object B in a third trial of the object recognition test regarding OVX rats in a control group and three experimental groups, in which raw dough without diosgenin was administered daily to the OVX rats in the control group; diosgenin-containing pellets (prepared using diosgenin and raw dough), which respectively comprised three daily dosages of diosgenin (i.e., 10, 50, and 100 mg/kg), were respectively administered to the OVX rats in diosgenin groups 1-3 daily; and the symbol “*” represents p<0.05.

### DETAILED DESCRIPTION OF THE PREFERRED EMBODIMENTS

it is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described. For clarity, the following definitions are used herein.

As used herein, the term “menopause” refers to the time from the beginning of the failure in the ovarian function to the cessation of menstruation, and the term “postmenopause” refers to the time after the cessation of menstruation.

As used herein, the term “menopausal syndrome” refers to symptoms occurring in menopausal or postmenopausal female subjects, including, but not limited to, physiological symptoms (e.g., hot flushes, vaginal dryness, fatigue, headaches, weight gain, skin aging, and osteoporosis), psychological symptoms (e.g., anxiety and depression), and cognitive deficits (e.g., mental confusion, decline in memory, learning, and recognition abilities, and inability to cope with stress).

Normally, health and daily life of menopausal or postmenopausal females are seriously influenced by menopausal syndrome. Thus, in recent years, numerous drugs have been developed so as to ameliorate different symptoms associated with menopausal syndrome.

It has been known that diosgenin is effective in ameliorating osteoporosis induced by menopause and in restoring keratinocyte proliferation in aged skin. In order to further investigate the effects of diosgenin on menopausal syndrome, the applicants used ovariectomized (OVX) rats as an animal model of menopause or postmenopause and conducted two experiments.

The first experiment is described as follows. The anxiety-like behavior of OVX rats was assessed by using the elevated plus-maze test, and was subsequently utilized to divide the OVX rats into two groups, namely, OVX rats with anxiety and OVX rats without anxiety. Diosgenin was administered to the OVX rats with and without anxiety, followed by evaluating the learning ability of the OVX rats via the learned helplessness test. The results of the first experiment verify that diosgenin is capable of significantly improving the learning ability of the OVX rats with anxiety.

By using the object recognition test conducted in the second experiment, the applicants found that administration of diosgenin is able to improve the ability to memorize a familiar object and the ability to recognize a difference between the familiar object and a novel object regarding OVX rats.

In view of the foregoing, diosgenin is expected to be capable of improving at least one of cognitive deficits associated with menopausal syndrome for females.

Accordingly, this invention provides a composition for improving at least one of cognitive deficits associated with menopausal syndrome, which comprises diosgenin. Preferably, the cognitive deficits are decline in learning, memory, and recognition abilities.

The composition according to this invention may be prepared in the form of a pharmaceutical composition or a food composition.

The pharmaceutical composition according to this invention can be formulated into a suitable dosage form for parenteral, topical, or oral administration using technology well known to those skilled in the art, which includes, but is not limited to, injections (e.g., sterile aqueous solutions or dispersions), sterile powder, tablets, troches, pills, capsules, and the like. In a preferred embodiment of this invention, the pharmaceutical composition is formulated into a suitable dosage form for oral administration.

The parenteral route of administration suitable for the pharmaceutical composition according to this invention includes, but is not limited to, intraperitoneal injection, subcutaneous injection, intramuscular injection, intravenous injection, sublingual administration, and transdermal administration.

The pharmaceutical composition according to this invention can additionally comprise a pharmaceutically acceptable carrier widely employed in the art of drug-manufacturing. For instance, the pharmaceutically acceptable carrier may include one or more of the following agents: solvents, emulsifiers, suspending agents, decomposers, binding agents, excipients, stabilizing agents, chelating agents, diluents, gelling agents, preservatives, lubricants, absorption delaying agents, liposomes, and the like.

The food composition according to this invention may be in the form of a food additive, which can be added into an edible material to prepare a food product for human or animal consumption. The food product according to this invention includes, but is not limited to, milk powder, a beverage, confectionery, a candy, a fermented food, a bakery product, animal feeds, a health food, and a dietary supplement.

The dosage and the frequency of administration of the composition according to this invention may vary depending on the following factors: the severity of the disease to be treated, the route of administration, and the weight, age, physical condition and response of the subject to be treated.

This invention also provides a method for improving at least one of cognitive deficits associated with menopausal syndrome in a subject, which comprises administering to the subject the aforementioned composition. Preferably, the cognitive deficits in the subject are decline in learning, memory, and recognition abilities.

In a preferred embodiment of this invention, the method is for improving the decline in learning ability of the subject that has anxiety. Preferably, a daily dosage of the aforementioned composition for improving the learning ability of the subject having anxiety may comprise a dosage of diosgenin ranging from 0.2 to 50 mg per kilogram of the body weight of the subject, may be administered in a single dose or in several doses, and may be orally administered. More preferably, the daily dosage of the aforementioned composition for improving the learning ability of the subject having anxiety may comprise the dosage of diosgenin ranging from 2 to 10 mg per kilogram of the body weight of the subject. Preferably, a daily dose of the aforementioned composition for improving the learning ability of the subject having anxiety and weighing 70 kg may comprise from 0.014 g to 3.5 g of diosgenin. More preferably, the daily dose of the aforementioned composition for improving the learning ability of the subject having anxiety and weighing 70 kg may comprise from 0.14 g to 0.7 g of diosgenin.

In another preferred embodiment of this invention, the method is for improving the decline in memory and recognition abilities of the subject. Preferably, a daily dosage of the aforementioned composition for improving the decline in memory and recognition abilities of the subject may comprise a dosage of diosgenin ranging from 1 to 100 mg per kilogram of the body weight of the subject, may be administered in a single dose or in several doses, and may be orally administered. More preferably, the daily dosage of the aforementioned composition for improving the decline in memory and recognition abilities of the subject may comprise the dosage of diosgenin ranging from 10 to 20 mg per kilogram of the body weight of the subject. Preferably, a daily dose of the aforementioned composition for improving the decline in memory and recognition abilities of the subject weighing 70 kg may comprise from 0.07 g to 7 g of diosgenin. More preferably, the daily dose of the aforementioned composition for improving the decline in memory and recognition abilities of the subject weighing 70 kg may comprise from 0.7 g to 1.4 g of diosgenin.

This invention will be further described by way of the following examples. However, it should be understood that the following examples are solely intended for the purpose of illustration and should not be construed as limiting the invention in practice.

### EXAMPLES

### Experimental Materials:

### 1. Experimental Animals:

Female Wistar rats (8 weeks of age, a body weight of about 264±2 g) purchased from National Laboratory Animal Center (R.O.C.) were used in Example 1, and female Wistar rats (8 weeks of age, a body weight of about 250±5 g) purchased from BioLasco Taiwan Co., Ltd. were used in Example 2. All of the experimental animals were kept in acrylic cages (35 cm×56 cm×19 cm) in an animal room with an independent air conditioning system under the following laboratory conditions: a temperature of 23±1° C., a relative humidity of 55±5%, and a 12 hour light/12 hour dark cycle (lights on at 7:00 am). Furthermore, food and water were provided ad libitum for all of the experimental animals.

Prior to an experiment, each of the experimental animals was handled so as to reduce stress responses thereof to an experimenter and an experimental environment. After the light was turned on for two hours, each of the experimental animals was placed in a clean cage and was delivered to an observation room having illuminance of 28˜30 lux for the experiment. The experimenter was required to leave the observation room so as to avoid interacting with the experimental animals during the experiment. All experimental procedures involving the experimental animals were approved by Animal Care Committee of Chung Shan Medical University and were performed in accordance with the NIH (National Institutes of Health) Guide for the Care and Use of Laboratory Animals.

### 2. Diosgenin-Containing Pellets:

Three daily dosages of diosgenin, i.e., 10, 50, or 100 mg/kg, were administered to the experimental animals in the following experiments. A proper amount of diosgenin (Sigma, USA, required for formulating a respective one of the three daily dosages) was mixed with 1 g of toast or raw dough (prepared by mixing flour and water in a ratio of 2:1 (wt/wt)), thereby forming a diosgenin-containing pellet. The experimental animals were orally administered with the diosgenin-containing pellets, starting about 4 weeks after ovariectomy.

### 3. Elevated Plus-Maze:

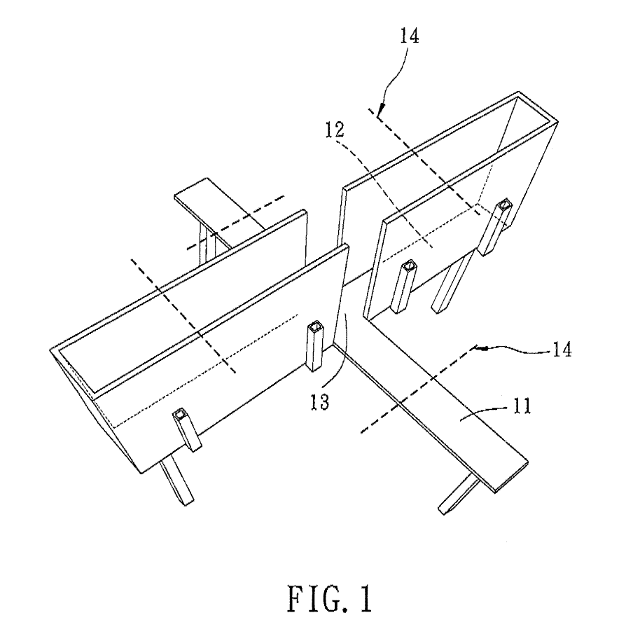

An elevated plus-maze used in Example 1 is shown in FIG. 1. The elevated plus-maze is generally in the shape of a cross, and is composed of two opposite open arms 11 (50 cm×10 cm), two opposite enclosed arms 12 (50 cm×10 cm×40 cm) that have side walls and no roofs, and an open square 13 (10 cm×10 cm) in the center. The elevated plus-maze was raised 50 cm above the floor. Each of open arms 11 and enclosed arms 12 is divided into a proximal half (relatively close to the open square 13) and a distal half (relatively far from the open square 13) by a virtual line 14. The elevated plus-maze was thoroughly cleaned using 20% ethanol and was subsequently thoroughly dried before each of the experimental animals was tested.

### 4. Shuttle Box:

A shuttle box used in Example 1 (AccuScan, USA) is composed of two equal compartments. Each of the compartments has a grid floor made of stainless steel bars. The compartments are separated by a wall having a central door. Specifically, the central door can be closed to separate the two compartments, or can be opened to allow the experimental animals to move freely between the two compartments therethrough. A conditioned stimulus (CS, a stimulus consisting of a tone (75 db) and a light (250 lux) and lasting 3 seconds) and an unconditioned stimulus (UCS, an electric foot shock (0.5 mA) lasting a maximum of 10 seconds) were administered to the experimental animals from the shuttle box under control of a computer. The shuttle box was thoroughly cleaned using 20% ethanol and was subsequently thoroughly dried before each of the experimental animals was tested.

### 5. Open Box:

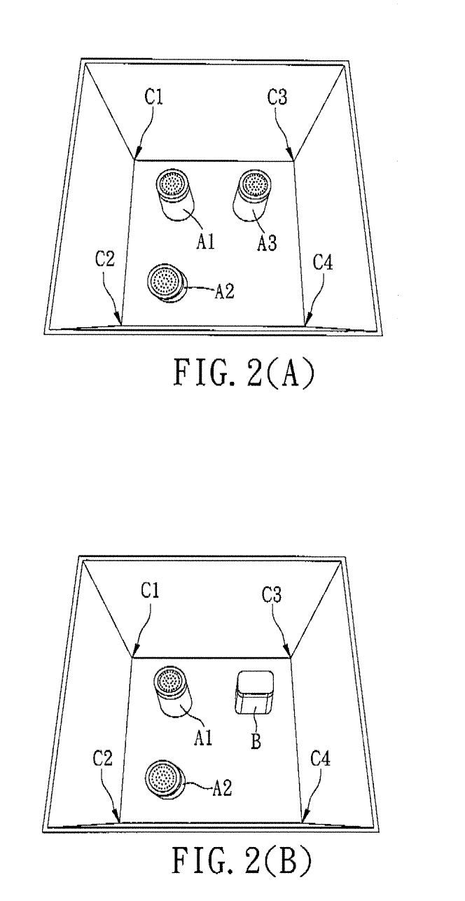

An open box used in Example 2 (60 cm×60 cm×40 cm) is shown in FIGS. 2(A) and 2(B). The open box is made of black polyvinyl plastics. Three objects, which are respectively designated as A1, A2, and A3, are identical in terms of size, color, shape, and material, and have no specific scents. Each of the objects A1, A2, A3 was placed on the bottom of the open box and was spaced apart from a respective one of three corners C1, C2, C3 of the open box by 21 cm (see FIG. 2(A)). The object A3 was later replaced with an object B different from the object A3 in terms of size, color, shape, and material, and having no specific scents (see FIG. 2(B)). Prior to an experiment, each of the objects A1, A2, A3, B was unfamiliar to the experimental animals. The open box and the objects A1, A2, A3, B were thoroughly cleaned using 20% ethanol, and were subsequently thoroughly dried before each of the experimental animals was tested.

### General Experimental Procedure:

### 1. Ovariectomy

After the experimental animals were raised in the animal room for four weeks, the same were subjected to ovariectomy, which was conducted according to the method as described in Y. J. Ho et al. (2007), supra, except for the following: the experimental animals were anesthetized by virtue of intramuscular injection of ketamine (100 mg/kg) or zoletil (2 mg/kg); skin of the experimental animals was incised using a surgical knife to expose the peritoneum under the skin, and an incision of about 1 cm was made in the peritoneum using the surgical knife; and the wounds of the OVX experimental animals housed individually in plastic cages were allowed to heal for about 5 days, and the OVX experimental animals were subsequently randomly grouped in home cages thereof.

### Example 1

### Effect of Diosgenin on Learning Ability of OVX Rats with Anxiety

A. Screening for OVX Rats with Anxiety

The female Wistar rats were randomly divided into two groups including an experimental group (n=74) and a sham-operated group (n=19). The rats in the experimental group were subjected to ovariectomy according to the method as described in the section, entitled “1. Ovariectomy”, of the General Experimental Procedure. The rats in the sham-operated group were subjected to a similar surgical procedure compared to the rats in the experimental group, except that the ovaries of the rats in the sham-operated group were not removed.

On the 29^{th} day after the surgery, the rats in each group were subjected to an elevated plus-maze (EPM) test to explore the effect of ovariectomy on the behavior of the rats, and to determine the presence or absence of anxiety symptoms for the rats.

The EPM test in this example was conducted substantially according to the method as described in Y. J. Ho at al. (2007), supra, except for minor modifications. The EPM test in this example is generally described as follows. A respective one of the rats of each group was placed in the open square 13 of the EPM and towards one of the two open arms 11 (see FIG. 1 for the EPM), and behavior thereof was observed and recorded for 5 minutes. A video camera installed above the EPM was used to monitor and record the behavior of each rat such that the open arm time, the enclosed arm time, the open arm activity, the enclosed arm activity, and the total arm activity thereof could be determined. The open arm time is defined as the time spent by an experimental animal in the open arms 11. The enclosed arm time is defined as the time spent by an experimental animal in the enclosed arms 12. The open arm activity is defined as the number of times that an experimental animal crosses the virtual lines 14 dividing the open arms 11. The enclosed arm activity is defined as the number of times that an experimental animal crosses the virtual lines 14 dividing the enclosed arms 12. The total arm activity is defined as the sum of the open arm activity and the enclosed arm activity.

The open arm time, the enclosed arm time, the open arm activity, the enclosed arm activity, and the total arm activity with respect to the rats in the sham-operated group were subjected to statistical processing. The thus obtained experimental data are expressed as mean±SEM. Afterward, the mean value of the open arm time with respect to the rats in the sham-operated group served as a reference value. In the experimental group, the rats were considered as having anxiety symptoms when the open arm time of the rats was lower than the reference value. On the contrary, in the experimental group, when the open arm time of the rats was higher than the reference value, the rats were considered as having no anxiety symptoms. Accordingly, the applicants further divided the rats in the experimental group into the OVX rats with anxiety and the OVX rats without anxiety. The open arm time, the enclosed arm time, the open arm activity, the enclosed arm activity, and the total arm activity regarding the OVX rats with and without anxiety were subjected to statistical processing. The resultant experimental data are expressed as mean±SEM.

The experimental data of each group were analyzed using t-tests so as to evaluate the difference between the OVX rats with anxiety and the rats in the sham-operated group, the difference between the OVX rats without anxiety and the rats in the sham-operated group, and the difference between the OVX rats with and without anxiety. Statistical significance is indicated by p<0.05.

### Results:

As set forth above, based on the open arm time compared with the reference value of the sham-operated group, the rats in the experimental group were divided into OVX rats with anxiety and the OVX rats without anxiety. Referring to Table 1, the open arm activity of the OVX rats with anxiety is significantly lower than that of the rats in the sham-operated group and the OVX rats without anxiety, and the enclosed arm time and the enclosed arm activity of the OVX rats with anxiety are significantly higher than those of the rats in the sham-operated group and the OVX rats without anxiety. In addition, the enclosed arm time of the OVX rats without anxiety is significantly lower than that of the rats in the sham-operated group. The aforesaid experimental results reveal that ovariectomy may cause a part of rats to have anxiety symptoms.

TABLE 1
Behavior of rats in experimental group and
in sham-operated group

| | | Experimental group | |
|---|---|---|---|
| | Sham-operated | OVX rats | OVX rats |
| | group | with anxiety | without anxiety |
| | (n = 19) | (n = 48) | (n =26) |
| Enclosed arm | 149.8 ± 15.3 | 205.9 ± 6.0***^{###} | 112.7 ± 5.4** |
| time | | | |
| (second) | | | |
| Open arm time | 99.3 ± 12.5 | 52.3 ± 4.4***^{###} | 130.6 ± 5.0** |
| (second) | | | |
| Enclosed arm | 16.6 ± 1.3 | 23.0 ± 0.9***^{###} | 17.9 ± 0.9 |
| activity | | | |
| (number of | | | |
| times) | | | |
| Open arm | 12.2 ± 1.8 | 6.0 ± 0.6***^{###} | 14.4 ± 0.8 |
| activity | | | |
| (number of | | | |
| times) | | | |
| Total arm | 28.8 ± 2.2 | 29.0 ± 1.1 | 32.3 ± 1.6 |
| activity | | | |
| (number of | | | |
| times) | | | |

**p < 0.01 when the OVX rats without anxiety are compared to the rats in the sham-operated group.
***p < 0.001 when the OVX rats with anxiety are compared to the rats in the sham-operated group.
^{###}p < 0.001 when the OVX rats with anxiety are compared to the OVX rats without anxiety.

### B. Effect of Diosgenin on Learning Ability of OVX Rats

The OVX rats with and without anxiety, which were identified in the preceding section A, were respectively randomly subdivided into 4 groups including a control group and three experimental groups (respectively designated as diosgenin groups 1-3). The diosgenin-containing pellets (made using diosgenin and toast), which were prepared according to the method as described in the section entitled “2. Diosgenin-containing pellets” of the Experimental Materials, respectively comprised the three daily dosages of diosgenin (i.e., 10, 50, and 100 mg/kg), and were respectively administered to the OVX rats in diosgenin groups 1-3 daily. In other words, the daily dosage of diosgenin administered to the OVX rats in diosgenin group 1 was 10 mg/kg, the daily dosage of diosgenin administered to the OVX rats in diosgenin group 2 was 50 mg/kg, and the daily dosage of diosgenin administered to the OVX rats in diosgenin group 3 was 100 mg/kg. The toast without diosgenin was administered daily to the OVX rats in the control group. After 23 days of administration, the OVX rats in each group were subjected to the following learned helplessness test so as to investigate the effect of diosgenin on the learning ability of the OVX rats with and without anxiety under stress.

The learned helplessness test in this example was conducted substantially according to the methods as described in W. F. Wang et al. (2007), Chin. J. Physiol., 50:63-68 and Y. T. Lee et al. (2008), Neuroreport, 19:1243-1247, except for minor modifications. The learned helplessness test in this example is generally described as follows.

### (1) Day-1 Session, Inescapable Foot Shock:

The respective OVX rat was placed in one of the two compartments of the shuttle box, and was allowed to explore the two compartments through the central door, which was opened, for 1 minute. Afterward, the central door was closed. The respective OVX rat was subjected to 40 trials of inescapable CS-UCS pairing. Specifically, each of the 40 trials of inescapable CS-UCS pairing consisted of a CS and a subsequent UCS, and a mean interval between the two successive trials of inescapable CS-UCS pairing was 60 seconds (the interval between the two successive trials of inescapable CS-UCS pairing ranged from 50 to 70 seconds).

### (2) Day-2 Session, Escapable Foot Shock (i.e., Active Avoidance Test):

The respective OVX rat was placed in one of the two compartments of the shuttle box, and was allowed to explore the two compartments through the central door, which was opened, for 1 minute. Subsequently, the respective OVX rat was subjected to 16 trials of escapable CS-UCS pairing. Specifically, each of the 16 trials of escapable CS-UCS pairing consisted of a CS and one of the following three options depending on a response of the respective OVX rat to the CS.

First, if the respective OVX rat moved into the adjoining compartment through the central door, which was opened, during the CS in the present trial of escapable CS-UCS pairing, the CS in the present trial of escapable CS-UCS pairing was immediately ceased, and no further UCS was transmitted. The present trial of escapable CS-UCS pairing was considered finished, and an avoidance response was recorded. After an interval of about 60 seconds, the next trial of escapable CS-UCS pairing was initiated.

Secondly, if the respective OVX rat failed to move into the adjoining compartment through the central door, which was opened, during the CS in the present trial of escapable CS-UCS pairing, a UCS was initiated immediately after the CS in the present trial of escapable CS-UCS pairing was over. If the respective OVX rat moved into the adjoining compartment through the central door, which was opened, during the UCS in the present trial of escapable CS-UCS pairing, the present trial of escapable CS-UCS pairing was considered finished. An escape response was recorded. After an interval approximately ranging from 47 to 57 seconds, the next trial of escapable CS-UCS pairing was initiated.

Thirdly, if the respective OVX rat failed to move into the adjoining compartment through the central door, which was opened, during the CS and 10 seconds of the UCS in the present trial of escapable CS-UCS pairing, the present trial of escapable CS-UCS pairing was considered finished after the UCS in the present trial of escapable CS-UCS pairing was over. A failure response was recorded. After an interval of about 47 seconds, the next trial of escapable CS-UCS pairing was initiated.

The number of avoidance response, the number of escape response, and the number of failure response in the 16 trials of escapable CS-UCS pairing were calculated. If the number of avoidance response of the OVX rats in a respective one of diosgenin groups 1-3 is higher than that of the OVX rats in the control group, or the number of escape response of the OVX rats in the respective one of diosgenin groups 1-3 is lower than that of the OVX rats in the control group, it is considered that diosgenin is able to facilitate learning of the OVX rats to make a shuttling response to avoid an electric shock.

The experimental data are expressed as mean±SEM. The experimental data were subjected to one-way analysis of variance (ANOVA) and were subsequently subjected to least-significant difference (LSD) test such that the difference between the control group and a respective one of diosgenin groups 1-3 could be evaluated. Statistical significance is indicated by p<0.05,

### Results:

Referring to Table 2, considering the OVX rats with anxiety, the number of avoidance response of the OVX rats in diosgenin group 1 is higher than that of the OVX rats in the control group, the number of escape response of the OVX rats in each of diosgenin groups 1 and 2 is lower than that of the OVX rats in the control group, and the number of escape response of the OVX rats in diosgenin group 1 is the lowest. Additionally, considering the OVX rats without anxiety, the number of avoidance response and the number of escape response regarding the OVX rats in each of diosgenin groups 1-3 are not obviously different from those regarding the OVX rats in the control group. The foregoing experimental results show that diosgenin is capable of improving the learning ability of the OVX rats with anxiety.

TABLE 2
Effect of diosgenin on behavioral response of OVX rats with and without anxiety in the avoidance test

| | OVX rats with anxiety (n = 48) | | | | OVX rats without anxiety (n = 26) | | | |
|---|---|---|---|---|---|---|---|---|
| | control | diosgenin | diosgenin | diosgenin | control | diosgenin | diosgenin | diosgenin |
| | group | group 1 | group 2 | group 3 | group | group 1 | group 2 | group 3 |
| | (n = 14) | (n = 12) | (n = 11) | (n = 11) | (n = 7) | (n = 7) | (n = 6) | (n = 6) |
| Avoidance | 1.7 ± 0.3 | 2.5 ± 0.9 | 1.3 ± 0.5 | 1.4 ± 0.4 | 1.0 ± 0.4 | 1.3 ± 0.6 | 1.2 ± 0.4 | 2.5 ± 0.8 |
| response | | | | | | | | |
| (number) | | | | | | | | |
| Escape | 12.3 ± 0.7 | 9.3 ± 1.3^{#} | 10.4 ± 1.4 | 13.2 ± 0.6 | 11.7 ± 1.7 | 11.7 ± 1.4 | 13.8 ± 0.5 | 12.5 ± 1.1 |
| response | | | | | | | | |
| (number) | | | | | | | | |
| Failure | 1.9 ± 0.8 | 4.2 ± 1.3 | 4.4 ± 1.4 | 1.4 ± 0.5 | 2.9 ± 1.6 | 3.0 ± 1.6 | 0.7 ± 0.3 | 0.8 ± 0.7 |
| response | | | | | | | | |
| (number) | | | | | | | | |

^{#}Considering the OVX rats with anxiety, p < 0.05 when the OVX rats in a respective one of diosgenin groups 1-3 are compared to the OVX rats in the control group.

### Example 2

### Effect of Diosgenin Upon Recognition and Memory Abilities of OVX Rats

In order to examine whether diosgenin is effective in improving recognition and memory abilities of OVX rats, the following experiment was performed.

After the female Wistar rats were raised in the animal room for 4 weeks, the same were subjected to ovariectomy according to the method as described in the section, entitled “1. Ovariectomy”, of the General Experimental Procedure. On the 29^{th} day after the surgery, the OVX rats were randomly divided into four groups including a control group (n=20) and three experimental groups (respectively designated as diosgenin group 1 (n=20), diosgenin group 2 (n=12), and diosgenin group 3 (n=19)). The diosgenin-containing pellets (made using diosgenin and raw dough), which were prepared according to the method as described in the section entitled “2. Diosgenin-containing pellets” of the Experimental Materials, respectively comprised the three daily dosages of diosgenin, i.e., 10, 50, and 100 mg/kg, and were respectively administered to the OVX rats in diosgenin groups 1-3 daily. In other words, the daily dosage of diosgenin administered to the OVX rats in diosgenin group 1 was 10 mg/kg, the daily dosage of diosgenin administered to the OVX rats in diosgenin group 2 was 50 mg/kg, and the daily dosage of diosgenin administered to the OVX rats in diosgenin group 3 was 100 mg/kg. The raw dough without diosgenin was administered daily to the OVX rats in the control group. After 26 days of administration, the OVX rats in each group were subjected to the following object recognition test.

The object recognition test in this example was conducted substantially according to the method as described in Wang W F et al. (2009), Behav. Neurosci., 123:1261-1270, except for minor modifications. One day prior to the object recognition test, the respective OVX rat was placed in the open box and adjacent to a corner C4 thereof, and was allowed to explore the objects A1, A2, A3 for 5 minutes (see FIG. 2(A) for the open box and the objects A1, A2, A3) so that the respective OVX rat was capable of adapting to the environment inside the open box.

The object recognition test in this example is described as follows. The object recognition test in this example included three trials. Each of the three trials lasted 5 minutes, and the interval between the first and second trials, and that between the second and third trials were 15 minutes. At the beginning of each of the three trials, the respective OVX rat was placed in the open box and adjacent to the corner C4 thereof. During the first and second trials, the objects A1, A2, A3 were disposed in the open box for being able to be explored by the respective OVX rat. During the third trial, the object A3 was replaced with the object B (see FIG. 2(E)). During the object recognition test, a video camera installed above the open box was used to monitor and record the behavior of the respective OVX rat. By virtue of the video camera, the time spent by the respective OVX rat exploring the object A3 in the second trial (referred to as exploration time spent on object A3 in the second trial) and the time spent by the respective OVX rat exploring the object B in the third trial (referred to as exploration time spent on object B in the third trial) were measured. Exploration of an object is defined as approaching the object and physically contacting the object with a snout and/or a forepaw. The difference between exploration time spent on object A3 in the second trial and exploration time spent on object B in the third trial can be used as an index for evaluating the ability to memorize a familiar object, and the ability to recognize the dissimilarity between the familiar object and a novel object.

The experimental data are expressed as mean±SEM. The experimental data were analyzed using paired-samples t-tests so as to evaluate the difference between exploration time spent on object A3 in the second trial and exploration time spent on object B in the third trial regarding the OVX rats in the respective group. Statistical significance is indicated by p<0.05.

### Results:

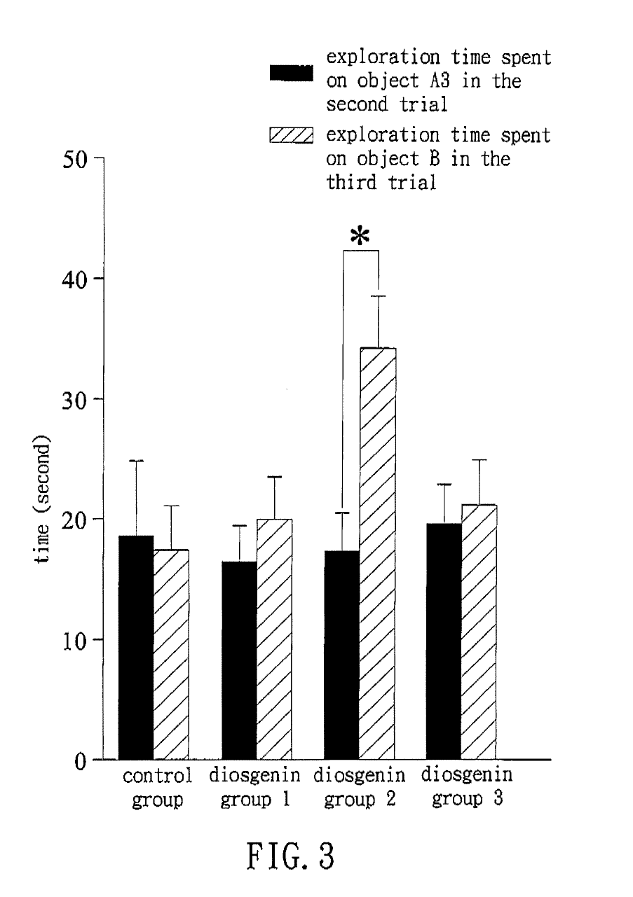

FIG. 3 shows exploration time spent on object A3 in the second trial and exploration time spent on object B in the third trial regarding the OVX rats in the control group and the three experimental groups. As shown in FIG. 3, considering the OVX rats in each of diosgenin groups 1-3, exploration time spent on object B in the third trial is longer than exploration time spent on object A3 in the second trial. In particular, exploration time spent on object B in the third trial regarding the OVX rats in diosgenin group 2 is the longest. The experimental results of Example 2 reveal that diosgenin is able to improve the ability to memorize a familiar object, and the ability to recognize a difference between the familiar object and a novel object, for the OVX rats.

All patents and literature references cited in the present specification as well as the references described therein, are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claims.

What is claimed is:

1. A composition for improving at least one of cognitive deficits associated with menopausal syndrome, the composition comprising diosgenin.

2. The composition as claimed in claim 1, wherein the cognitive deficits are decline in learning, memory, and recognition abilities.

3. The composition as claimed in claim 1, which is for improving the decline in learning ability.

4. The composition as claimed in claim 1, which is for improving the decline in memory and recognition abilities.

5. The composition as claimed in claim 1, which is a pharmaceutical composition.

6. The composition as claimed in claim 5, which is in an oral dosage form.

7. The composition as claimed in claim 1, which is a food composition.

8. A method for improving at least one of cognitive deficits associated with menopausal syndrome in a subject, the method comprising administering to the subject a composition as claimed in claim 1.

9. The method as claimed in claim 8, wherein the cognitive deficits are decline in learning, memory, and recognition abilities.

10. The method as claimed in claim 8, wherein the composition is a pharmaceutical composition.

11. The method as claimed in claim 10, wherein the composition is orally administered to the subject.

12. The method as claimed in claim 8, wherein the composition is a food composition.

13. The method as claimed in claim 9, which is for improving the decline in learning ability of the subject that has anxiety.

14. The method as claimed in claim 13, wherein a daily dosage of the composition comprises a dosage of diosgenin ranging from 0.2 to 50 mg per kilogram of a body weight of the subject.

15. The method as claimed in claim 9, which is for improving the decline in memory and recognition abilities of the subject.

16. The method as claimed in claim 15, wherein a daily dosage of the composition comprises a dosage of diosgenin ranging from 1 to 100 mg per kilogram of a body weight of the subject.
